(19) United States
(12) Patent Application Publication
(10) Pub. No.: US 2006/0183955 A1
(43) Pub. Date: Aug. 17, 2006

(54) Freeze dry process for the preparation of a high surface area and high pore volume catalyst

(75) Inventors: Xin Xiao, Houston, TX (US); James R. Butler, League City, TX (US)

Correspondence Address: David J. Alexander, P.O. Box 674412, Houston, TX, 77167, US

(73) Assignee: Fina Technology, Inc., Houston, TX (US)

(21) Appl. No.: 11/397,984

(22) Filed: Apr. 5, 2006

Related U.S. Application Data
(62) Division of application No. 10/442,773, filed on May 21, 2003.

(51) Int. Cl. C07C 5/13; B01J 21/14

(52) U.S. Cl. 585/750; 502/242

(57) ABSTRACT

The present invention provides a process for the preparation of a catalyst having a high surface area and pore volume. The process includes freeze drying an intermediary of the catalyst. The present invention further includes a catalyst prepared by a process that includes the freeze drying step. The present invention also includes a catalyst having a high acidity, as indicated by having an ammonium desorption peak at greater than about 500° C. The prevent invention further includes a method of manufacturing isomerized organic compounds using the catalyst.

### TECHNICAL FIELD OF THE INVENTION

The present invention is directed, in general, to a process for preparing a catalyst comprising a freeze drying step. The catalyst has a surface area of greater than about 40 m²/g and a pore volume of greater than about 0.1 ml/g. Moreover, the catalyst has high acidity, as indicated by a peak ammonia desorption at greater than about 500° C.

### BACKGROUND OF THE INVENTION

Solid acid catalysts are desirable over liquid phase acid catalysts in a number of respects, including reduced environmental burden for disposal, reduced corrosion of reactors and easier separation of products from the catalyst. Solid acid catalysts may also have superior stability and catalytic activity for a number of hydrocarbon conversions. To be used in a commercial setting, however, it is desirable to maximize the activity of the solid acid catalyst. However, certain acid catalysts having, for example, an aluminum chloride based support may be problematic due to their fragility, inactivation by water, oxygen or sulphur, the need for corrosive dopants to maintain activity and the inability to regenerate an inactivated catalyst. Moreover, such alumina supported catalysts may have low activity for certain reactions, such as the isomerization of paraffins.

Zirconium oxides have been suggested as alternative catalysts for the isomerization of paraffins, as well as other petrochemical and refinery applications. However, previous preparations of such catalysts have low catalytic activity or are otherwise unsuitable for industrial application. It is thought that the activity of zirconia based catalysts may be improved by increasing the surface area and pore volume of the catalyst's structure. The surface area and pore volume provide active sites and access of reactants to the active sites.

Certain steps in the manufacture of zirconium oxide catalyst, and the sequence of such steps, have been proposed to be important in controlling the porosity of the catalyst. Such steps may include the process for the deposit of hydrated zirconia of a support, calcination, sulphation, the deposit of a hydrogenating transition metal, and the washing and drying of intermediaries. For example, depositing a hydrated zirconia on a support such as alumina or silica by impregnation of the support with a zirconium salt solution may be followed by drying for several hours at an elevated temperature, such as 120° C. Or, the precipitation of a zirconium salt solution with a base, either before or after mixing with a refractory mineral, such as alumina or silica, may be followed by washing the precipitate with water or a polar organic solvent, and drying for several hours at an elevated temperature, such as 60° C. or 120° C.

Such drying processes, however, may not be conducive to the optimal plant scale production of acid catalyst having high activity. For example, drying intermediaries by heating for several hours may be inefficient both in terms of time and energy utilization. And, the handling and removal of organic solvents may require costly alterations to existing catalyst production facilities. Moreover, such drying procedures may not facilitate the optimal production of high surface area and pore volume acid catalysts.

Accordingly, what is needed is a process for drying solid acid catalysts that is conducive to both the commercial production of such catalysts and the production of catalysts having a high surface area and pore volume, and a high acidity, while not experiencing the above-mentioned problems.

### SUMMARY OF THE INVENTION

To address the above-discussed deficiencies, the present invention provides, in one embodiment, a process for the preparation of a catalyst comprises preparing an intermediate of a catalyst and freeze drying the intermediary. Another embodiment of the present invention provides a catalyst prepared by a process comprising the above-mentioned freeze drying step.

In yet another embodiment, the present invention provides a catalyst having a peak ammonia desorption of greater than about 500° C. Still another embodiment is a method of manufacturing isomerized organic compounds using a catalyst prepared by a process comprising freeze drying an intermediary of the catalyst. The method further includes contacting an organic compound with the catalyst under conditions sufficient to allow isomerization of the organic compound.

The foregoing has outlined preferred and alternative features of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention. Those skilled in the art should also realize that such equivalent constructions do not depart from the spirit and scope of the invention.

### BRIEF DESCRIPTION OF THE DRAWINGS

For a more complete understanding of the invention, reference is now made to the following descriptions taken in conjunction with the accompanying drawing, in which:

FIG. 1 illustrates the BJH-DFT analysis results of pore volume distribution with respect to pore diameter for tungstated zirconium oxide (WZ) prepared by a step comprising 110° C. drying or freeze drying;

FIG. 2 illustrates the BJH-DFT analysis results of surface area distribution with respect to pore diameter for tungstated zirconium oxide (WZ) prepared by a step comprising 110° C. drying or freeze drying;

FIG. 3 illustrates the BJH-DFT analysis results of pore volume distribution with respect to pore diameter for sulphated zirconium oxide (SZ) prepared by a step comprising 110° C. drying or freeze drying;

FIG. 4 illustrates the BJH-DFT analysis results of surface area distribution with respect to pore diameter for sulphated zirconium oxide (SZ) prepared by a step comprising 110° C. drying or freeze drying; and

FIG. 5 illustrates the ammonia desorption of sulphated zirconium oxide prepared in the presence of colloidal silica and subsequent freeze dry (SZ-silica), and sulphated zirconium oxide prepared in the presence of colloidal silica and subsequent freeze dry plus Fe and Mn (FeMn SZ-silica).

FIG. 6 illustrates the BJH-DFT analysis results of pore volume distribution with respect to pore diameter for sulphated zirconium oxide prepared in the presence of colloidal silica and subsequent freeze dry (SZ-silica) and sulphated zirconium oxide prepared in the presence of colloidal silica and subsequent freeze dry and Fe plus Mn (FeMn SZ-silica); and

FIG. 7 illustrates the BJH-DFT analysis results of surface area distribution with respect to pore diameter for sulphated zirconium oxide prepared in the presence of colloidal silica and subsequent freeze dry (SZ-silica) and sulphated zirconium oxide prepared in the presence of colloidal silica and subsequent freeze dry and Fe plus Mn (FeMn SZ-silica).

### DETAILED DESCRIPTION

The present invention discloses the hitherto unrecognized ability of a freeze drying step to facilitate the production of a catalyst having a high surface area and pore volume. This, in turn, should allow for the more cost efficient plant-scale production of catalysts having high activity. In certain preferred embodiments, the catalyst may be a solid acid catalyst. The catalyst particles may be shaped into any form commonly used for the industrial implementation of solid catalysts, for example, beads, extrusions, and pellets.

The present invention is directed to a process for the preparation of a catalyst comprising freeze drying an intermediary of a catalyst. The term intermediary as used herein refers to the precipitate resulting when a solution containing a catalyst precursor, such as a Group IV salt, is mixed with a base. For example, a zirconium hydroxide may be the intermediary resulting when a solution containing zirconyl chloride is precipitated by adding ammonia to the solution. Other non limiting examples of Group IV salts include zirconium tetrachloride, zirconium nitrate, zirconyl sulphate and zirconium sulphate. Other examples include hafnium and titanium metal cations in combination with any of the above-mentioned anions. In certain preferred embodiments the intermediary is isolated by filtering or centrifuging the neutralized solution containing the Group IV salt and base, resulting in a solid cake, which may then be freeze dried.

Freeze drying may be carried out using any conventional apparatus capable of drawing a vacuum for a period sufficient to remove substantially all the free water from the intermediary. The chemical bonded water precursor, as the form of hydroxyl group, is preserved. For example, a commercial freeze dryer typically used in food processing would be suitable. Preferably, the vacuum is less than about 100 mTorr and more preferably less than about 10 mTorr. Preferably, the freeze dryer may maintain the temperature of the intermediary at less than about 0° C., and more preferably less than about −20° C. In certain embodiments, the process may further include freezing the intermediary prior to freeze drying.

After freeze drying the catalyst may be stored or loaded into an industrial reactor without taking any further processing steps. It is however preferable to calcinate it at high temperature, as discussed above, in a dry atmosphere before using it. The term calcination as used herein refers to heating the intermediary at a high temperature, preferably between about 400 and about 850° C., and more preferably about 650° C. for at least about 1 hour.

The process may further include calcinating the intermediary to obtain a catalyst having a surface area of greater than about 40 m²/g and a pore volume of at least about 0.10 ml/g. The terms pore volume and surface area distribution as used herein refer, respectively, to the pore volume and surface area measured for the entire range of pore diameters present in a catalyst. These parameters may be expressed as a total pore volume (PV) per gram of catalyst or total surface area (SA) per gram of catalyst, respectively, for example, as measured by conventional gas absorption techniques and using the Brunauer, Emmett and Teller model (BET). Or, the distributions of pore volumes and surface areas, over the range of pore diameters present in the support material, may be measured using conventional methods, such as the Barrett-Joyner-Halenda (BJH) method, and the Oliver-Conklin Density Function Theory (DFT).

The process may further include anion modification of the catalyst. The term anion-modified refers to the process whereby anions, such as sulphate or tungstate, are added to the intermediary prior to freeze drying. Anion modification is thought to increase the acidity of the catalyst. Anion modification may also help increase the surface area and pore volume of the catalyst by preserving the pore structure and preventing particle agglomeration during calcination.

In certain embodiments, the process of anion modification includes sulphation of the catalyst by adding sufficient amounts of any precursor of sulphate ions, such as ammonium sulfate or H₂SO₄, to the filtration cake to give about 2 to about 10 wt % of S in the catalyst (i.e., after calcination), and more preferably from about 3 to about 6 wt %. In other embodiments, the process includes tungstation of the catalyst by adding sufficient amounts of any precursor of tungstate ions, such as ammonium metatungstate ((NH₄)₆ H₂W₁₂O₄₀)), to the solid cake to give about 4 to about 30 wt % W in the catalyst, and more preferably about 12 to 18 wt %. Following calcination, the anion modified catalyst may have a surface area of greater than about 67 m²/g and a pore volume of at least about 0.12 ml/g, and more preferably a surface area of greater than 110 m²/g and a pore volume of at least about 0.16 ml/g.

The process of the present invention may give rise to a catalyst having very high acidity. For example, the process may include calcinating the intermediary to obtain a catalyst having a peak ammonia desorption of greater than about 500° C. And, as further illustrated in the Experimental section to follow, in certain preferred embodiments, the peak ammonia desorption may be at least about 600° C., and in other embodiments, at about 700° C. The term peak ammonia desorption as used herein refers to the temperature of maximum ammonium desorption obtained during conventional temperature program desorption experiments, as illustrated in the Experiment section to follow.

In certain embodiments of the process, sufficient base may be added to increase the pH to greater than about 6, and more preferably greater than about 8, and still more preferably greater than about 10 during precipitation. In other advantageous embodiments, the base comprises a volatile organic amine, for example, ammonium hydroxide or one or more amines containing five carbons or less, or combinations thereof. In certain preferred embodiments the base is a concentrated solution comprising, for example, 28 vol % ammonium hydroxide.

The process may further include aging the intermediary by heating it for a period. In certain embodiments aging may include maintaining the intermediary at between about 40 and about 110° C., and preferably, about 100° C., for greater than about 4 hours, and preferably about 16 to about 24 hours, after precipitation, but before freeze drying. In other embodiments, however, the aging step may be for about 40 hours, or longer. Following calcination of the aged intermediary, the catalyst may have a surface area of greater than about 80 m²/g and a pore volume of at least about 0.25 ml/g, and more preferably a surface area of greater than 150 m²/g and a pore volume of at least about 0.27 ml/g.

The process may further include the intermediary comprising a refractory mineral. The term refractory mineral as used herein refers to any mineral oxide that may impart structural stability to the catalyst. Examples of suitable refractory minerals include aluminas, silicas, silica-aluminas, alumino-silicates, clays and combinations thereof. Preferably, the refractory mineral is added to the Group IV salt prior to precipitation with base. The refractory mineral preferably ranging from about 0.5 to about 10 wt % in the Group IV salt solution. For example, in certain preferred embodiments, colloidal silica may be added to zirconyl chloride to provide about 1.5 wt % in silica, and the mixture precipitated with base and further processed as discussed above.

As further illustrated in the Experimental section to follow, the inclusion of a refractory mineral may facilitate the production of a catalyst having a high surface area and pore volume. For example, following calcination of the refractory mineral containing intermediary, the catalyst may have a surface area of greater than about 82 m²/g and a pore volume of at least about 0.27 ml/g, and more preferably a surface area of greater than 146 m²/g and a pore volume of at least about 0.4 ml/g.

In an alternative advantageous embodiment, the process may include depositing a Group IV salt into a support. For example, the Group IV salt may be precipitated with base in the presence of a support, with subsequent freeze drying of the intermediary and support and other processing steps as described above. The support may comprise any material suitable for the preparation of a solid acid catalyst. The support may include, for example, silica, alumina, clays, magnesia, zeolite, active carbon, gallium, titanium, thorium, boron oxide and combinations thereof.

The process may further include the intermediary comprising a metal promoter. The term metal promoter refers to a Group VIIB or VIIIB metal, such as Fe or Mn. It is thought that such metal promoters help increase the activity of the catalyst. In certain preferred embodiments, the metal promoters comprise from about 0.05 wt % to about 5 wt % of the catalyst. As further illustrated in the Experimental section to follow, metal promoters may also facilitate the production of catalysts having a high surface area and pore volume, and having a high acidity.

Another embodiment of the present invention is directed to a catalyst prepared by the process the includes freeze drying an intermediary of a catalyst. In certain embodiments, the catalyst may comprise a Group IV oxide having a surface area of greater than about 40 m²/g and a pore volume of at least about 0.10 ml/g. In other preferred embodiments the catalyst may comprise an anion-modified Group IV oxide having a surface area of greater than about 60 m²/g and a pore volume of at least about 0.11 ml/g.

Any of the above-mentioned processing steps performed on the intermediary may be included in the process to prepare the catalysts of the present invention. For example, the catalyst may be prepared by process further including aging by maintaining the catalyst at about 110° C. for about 16 to about 24 hours following freeze drying. Such catalysts may comprise a Group IV oxide having a surface area of greater than about 73 m²/g and a pore volume of at least about 0.23 ml/g. Or, in other preferred embodiments, the catalyst may comprise an anion-modified Group IV oxide containing a refractory mineral and having a pore volume of at least about 0.27 ml/g.

Yet another embodiment of the present invention is directed to a catalyst having a peak ammonia desorption of greater than about 500° C. Such catalysts are thought to have high activity by virtue of their high acidity. In other preferred embodiments, the catalyst may have a peak ammonia desorption of about 600° C. In still other embodiments, the catalyst may have a peak ammonia desorption of greater than about 700° C. In certain preferred embodiments the catalyst may comprise an anion-modified Group IV oxide, for example, sulphated zirconium oxide. And, yet other preferred embodiments the catalyst may further include metal promoters, such as Fe and Mn. In such embodiments, the catalyst may further have a surface area of greater than about 140 m²/g and a pore volume of at least about 0.30 ml/g.

Still another embodiment of the present invention is directed to a method of manufacturing isomerized organic compounds. The method includes preparing a catalyst by a process comprising freeze drying an intermediary of said catalyst. The method further includes contacting an organic compound with said catalyst under conditions sufficient to allow isomerization of the organic compound. The organic compound may include paraffins having nine carbons or less or cyclic hydrocarbons having nine carbons or less. For example, a solid acid catalyst comprising any catalyst prepared as described above may be used to isomerise C₅ or C₆ paraffins, and thereby boost the octane rating of fuels containing such paraffins. Alternatively, the catalyst may be used in the isomerization of olefins or cyclical and aromatic compounds.

Moreover, the catalyst of the present invention may be used in any hydrocarbon transformation chemical reaction requiring the use of an acid. Such reactions may include alkylation, oligomerization, hydrocarbon dehydration or transformations by hydrocracking or hydroisomerization.

Having described the present invention, it is believed that the same will become even more apparent by reference to the following experiments. It will be appreciated that the experiments are presented solely for the purpose of illustration and should not be construed as limiting the invention. For example, although the experiments described below were carried out in a laboratory or pilot plant, one skilled in the art could adjust specific numbers, dimensions and quantities up to appropriate values for a full scale plant.

### EXPERIMENTS

Four experiments were conducted to examine the effect of freeze drying on the porosity solid acid catalysts and on the acidity of such catalysts.

### Experiment 1

One experiment was performed to evaluate the effect of freeze drying on the surface area (SA) and pore volume (PV) of a zirconium oxide containing catalyst in the presence and absence of anion modification and heat aging, as compared to other drying procedures. The analysis of the pore characteristics (i.e., pore volume, surface area, pore diameter and distributions) was conducted on an ASAP 2400 (Micromeritics Instrument Corp., Norcross, Ga.), using nitrogen as the adsorbate for the conventional measurements of adsorption and desorption isotherms. The data was used for the calculation, using the BET model of total surface area, total pore volume and average pore diameter. In addition, the data were analyzed to determine the pore volume and surface area distributions using the classical Kelvin equation, Harkins and Jura model and DFT PLUS software (Micromeriticus Instrument Corp., Norcross, Ga.).

Zirconyl chloride (ZrOCl₂ 8H₂O) was dissolved in deionized water and precipitated by adding an ammonium hydroxide solution (˜28 wt % ammonia in water) until the pH was about 9. The resulting slurry was divided into two lots. The first lot (“non-aged”) was filtered, washed and further processed as described below. The second lot was heat aged (“aged”) by maintaining the solution at about 100° C. with agitation for about 24 hours. The precipitated zirconium hydroxide (Zr(OH)₄) slurry from both the first and second lots were filtered and washed several times with deionized water. The filtrate (“m cake”) from each lot was then divided into three portions each and dried using one of three different methods further described below. After drying the portions were each further divided into three samples. Two samples were anion-modified by impregnating the filtration cake with either about 0.5 M H₂SO₄ or ammonium metatungstate (˜12 wt % tungsten). All three samples were then dried at about 110° C. for about 12 hours followed by calcination at about 650° C. for about 24 hours to produce Zirconium Oxide (ZrO₂), Sulphated Zirconium Oxide (SZ) and Tungstated Zirconium Oxide (WZ).

Separate portions of the filtration cakes were dried by either: (1) heating at about 110° C. at atmospheric pressure for about 12 hours (designated as, “110° C. drying” or “110° C. dried”); (2) suspending the filtration cake in acetone (cake:acetone ˜1:20) followed by filtration, and then repeating the suspension and filtration steps two more times before heating the cake portion at about 60° C. at atmospheric pressure for about 12 hours (designated as, “Ace/60° C. drying” or “Ace/60° C. dried”); and (3) freeze drying (“FD”) in a conventional freeze dryer (Ace Glass Inc., Vineland, N.J.) using a vacuum of less than about 10 mTorr for 24 hours. The freeze dryer was immersed in dry ice-acetone and a liquid nitrogen trap was used to protect the vacuum pump.

The portion of the filtration cake (about 10 ml to about 200 ml) that was subjected to freeze drying was placed into a glass flask and attached to the freeze dryer with no prior cooling of the cake. The cake was observed to freeze within about 5 minutes of attachment to vacuum. And, substantially all the water in the sample was removed within about 1 hour, as revealed by the absence of visible frost in the flask and by the powdery appearance of the dried cake.

The results of the BET analysis are summarized in TABLE 1. The freeze dried non-aged ZrO₂ sample had a higher surface area and pore volume as compared to 110° C. drying, and higher surface area as compared to Ace/60° C. drying. Aging generally increased the surface area and pore volume, with the same higher surface area and pore volume for the freeze dried as compared to 110° C. and 60° C. drying, as discussed above.

Anion modification also generally increased the surface area and pore volume of all samples. Non-aged and aged freeze dried SZ had a higher surface area than both 110° C. and 60° C. dried SZ. And freeze dried WZ had an improved surface area and pore volume than 110° C. dried WZ and a higher surface area than Ace/60° C. dried WZ.

TABLE 1

| | 110° C. | | Ace/60° C. | | FD | |
|---|---|---|---|---|---|---|
| | SA | PV | SA | PV | SA | PV |
| Sample | (m²/g) | (ml/g) | (m²/g) | (ml/g) | (m²/g) | (ml/g) |

Non-Aged

| | | | | | | |
|---|---|---|---|---|---|---|
| ZrO₂ | 28.3 | 0.09 | 38.5 | 0.23 | 56.8 | 0.13 |
| SZ | 77.0 | 0.09 | 107.4 | 0.26 | 110.7 | 0.16 |
| TZ | 84.1 | 0.10 | 81.1 | 0.24 | 68.2 | 0.12 |

Aged

| | | | | | | |
|---|---|---|---|---|---|---|
| ZrO₂ | 77.0 | 0.22 | 68.4 | 0.34 | 82.0 | 0.27 |
| SZ | 145.8 | 0.21 | 89.4 | 0.13 | 151.1 | 0.27 |
| TZ | 110.3 | 0.23 | 107.6 | 0.36 | 111.6 | 0.25 |

### Experiment 2

A second experiment further investigated the effect of freeze drying versus 110° C. drying on surface area and pore volume, when used in combination with other optional processing steps. The optional processing steps included the preparation of zirconium oxide in the presence and absence of anion modification (i.e., SZ and WZ) in combination with: heat aging for one of two different periods; the precipitation of zirconium hydroxide at one of two different pHs; and the presence and absence of a refractory mineral. All combinations of these steps were investigated for ZrO₂, SZ and WZ catalyst preparations.

The preparation of catalysts with different periods of aging proceeded similar to that described in Experiment 1 with the exception that the precipitate slurry was maintained at about 100° C. with agitation for either about 16 or about 40 hours. The preparation of catalysts at two different pHs (“pH”) was also similar to the process followed in Experiment 1 with the exceptions of the above-described modification to the aging step and the precipitation of Zirconyl chloride by adding the ammonium hydroxide solution until the pH was either about 8 or about 10. The preparation of catalysts in the presence of a refractory mineral (“Silica”) was also as described in Experiment 1, with the exceptions of the above-described modification to the aging procedure and precipitation steps, and the addition of about 1.5 wt % colloidal silica to the Zirconyl chloride before the precipitation step.

The surface area and pore volume of the different catalytic preparations were measured using the above-described BET methodology and DFT theory. In addition, pore diameter (PD), was calculated using the equation: PD=4PV/SA. The results of these measurements are summarized in TABLE 2.

TABLE 2

| | | | 110° C. Drying | | | | Freeze Drying | | |
|---|---|---|---|---|---|---|---|---|---|
| pH | Silica | Run No. | SA (m²/g) | PV (ml/g) | PD (Δ) | Run No. | SA (m²/g) | PV (ml/g) | PD (Δ) |

ZrO₂
Aged 16 hours

| | | | | | | | | | |
|---|---|---|---|---|---|---|---|---|---|
| 8 | no | 1 | 54 | 0.19 | 141 | 13 | 62.5 | 0.23 | 147 |
| 8 | yes | 4 | 74.8 | 0.17 | 91 | 16 | 82.1 | 0.32 | 156 |
| 10 | no | 7 | 81.5 | 0.22 | 108 | 19 | 75.6 | 0.26 | 138 |
| 10 | yes | 10 | 94.4 | 0.24 | 102 | 22 | 103.2 | 0.41 | 159 |

Aged 40 hours

| | | | | | | | | | |
|---|---|---|---|---|---|---|---|---|---|
| 8 | no | 25 | 55.7 | 0.21 | 151 | 37 | 52.7 | 0.25 | 190 |
| 8 | yes | 28 | 90.9 | 0.2 | 88 | 40 | 85 | 0.34 | 160 |
| 10 | no | 31 | 82.3 | 0.22 | 107 | 43 | 72.3 | 0.25 | 138 |
| 10 | yes | 34 | 104.3 | 0.25 | 96 | 46 | 101.3 | 0.41 | 162 |

SZ
Aged 16 hours

| | | | | | | | | | |
|---|---|---|---|---|---|---|---|---|---|
| 8 | no | 2 | 145 | 0.2 | 55 | 14 | 131.4 | 0.26 | 79 |
| 8 | yes | 5 | 165 | 0.18 | 44 | 17 | 138.8 | 0.31 | 89 |
| 10 | no | 8 | 147.8 | 0.21 | 57 | 20 | 140 | 0.26 | 74 |
| 10 | yes | 11 | 179.2 | 0.26 | 58 | 23 | 136.8 | 0.37 | 108 |

Aged 40 hours

| | | | | | | | | | |
|---|---|---|---|---|---|---|---|---|---|
| 8 | no | 26 | 160.5 | 0.23 | 57 | 38 | 131.2 | 0.28 | 85 |
| 8 | yes | 29 | 179.9 | 0.21 | 47 | 41 | 137.3 | 0.31 | 90 |
| 10 | no | 32 | 148.6 | 0.21 | 57 | 44 | 133.6 | 0.23 | 69 |
| 10 | yes | 35 | 174.9 | 0.26 | 59 | 47 | 134.8 | 0.35 | 104 |

WZ
Aged 16 hours

| | | | | | | | | | |
|---|---|---|---|---|---|---|---|---|---|
| 8 | no | 3 | 75.3 | 0.22 | 117 | 15 | 83 | 0.28 | 135 |
| 8 | yes | 6 | 135.5 | 0.26 | 77 | 18 | 117.7 | 0.33 | 112 |
| 10 | no | 9 | 91.4 | 0.23 | 101 | 21 | 104.3 | 0.29 | 111 |
| 10 | yes | 12 | 124.6 | 0.25 | 80 | 24 | 134.5 | 0.42 | 125 |

Aged 40 hours

| | | | | | | | | | |
|---|---|---|---|---|---|---|---|---|---|
| 8 | no | 27 | 72.5 | 0.24 | 132 | 39 | 90 | 0.33 | 147 |
| 8 | yes | 30 | 131.6 | 0.21 | 64 | 42 | 124.8 | 0.36 | 115 |
| 10 | no | 33 | 101.9 | 0.25 | 98 | 45 | 103.2 | 0.3 | 116 |
| 10 | yes | 36 | 120.6 | 0.18 | 60 | 48 | 146.6 | 0.44 | 120 |

### Experiment 3

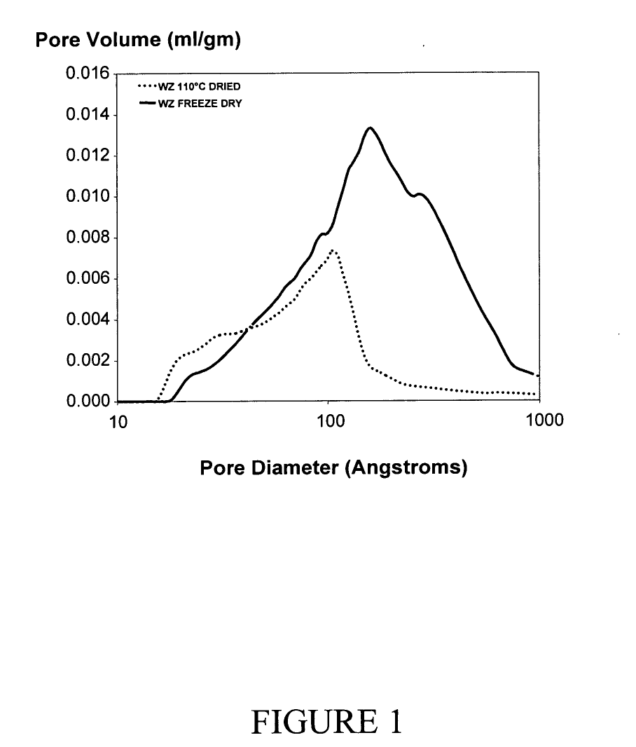

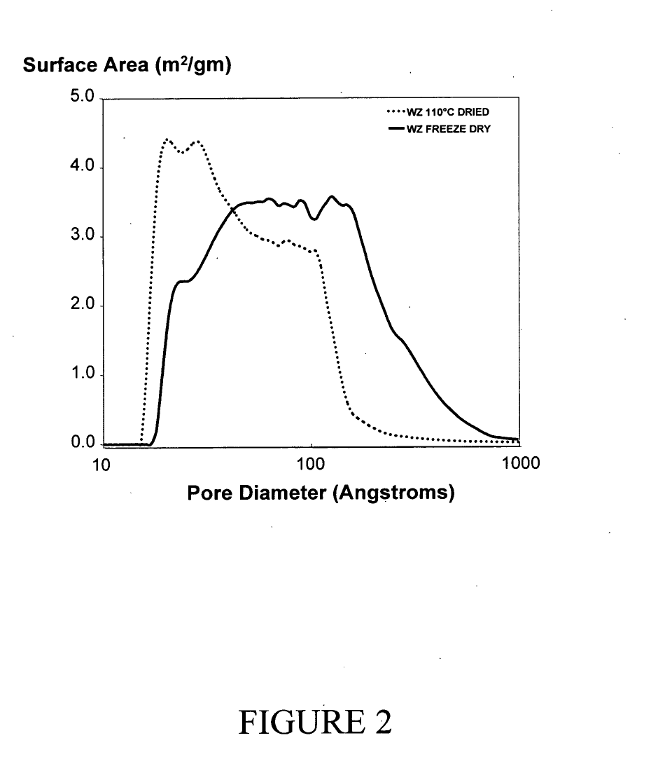

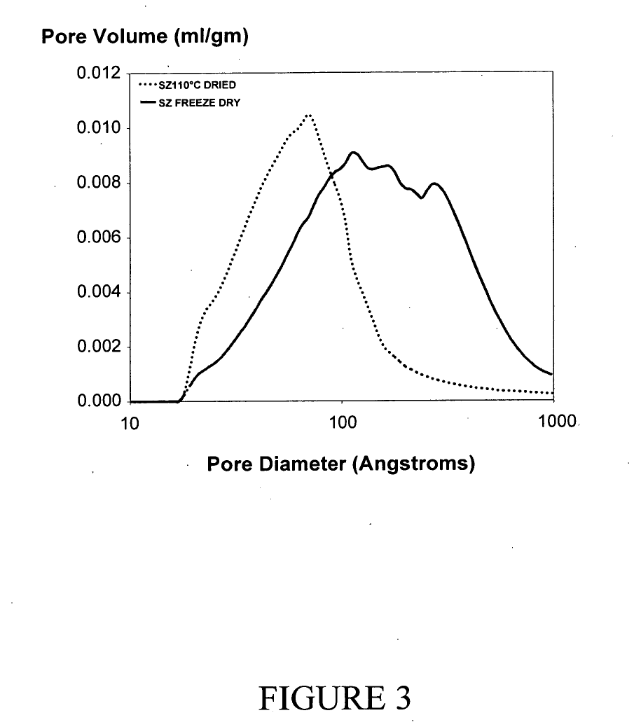

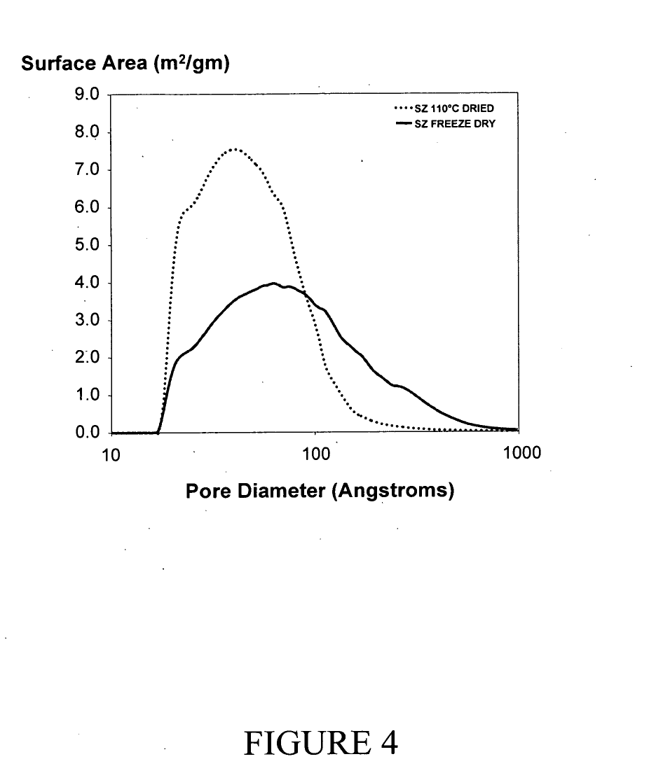

A third experiment compared the effect of freeze drying versus 110° C. drying on the pore size distribution of anion-modified zirconium oxide in the presence of a refractory mineral. WZ and SZ were prepared in the presence of 1.5 wt % colloidal silica and subject to BET and DFT analyses as described above. The distribution of pore volume and surface area over a range of pore diameters is depicted in FIGS. 1 and 2 (W) and FIGS. 3 and 4 (SZ). The surface area and pore volumes occurred at higher pore diameters for freeze dried as compared to 110° C. dried preparations of catalyst. For example, 110° C. dried WZ had a peak pore volume and surface area centered at about 105 Angstroms and about 24 Angstroms, respectively. In contrast, freeze dried WZ had a peak volume and surface area center at about 160 and about 97 Angstroms, respectively. Likewise, freeze dried SZ had peak surface area and pore volume centered at about 118 and about 62 Angstroms, whereas the analogous values for 110° C. dried SZ were about 70 and about 41 Angstroms, respectively.

### Experiment 4

A fourth experiment assessed the acidity of anion-modified zirconium oxide, prepared in the presence of colloidal silica using a freeze drying step, in the presence (“FeMnSZ-silica”) and absence of Fe and Mn promoters (“ZS-silica”). The effect of the two promoters on pore size distribution was also examined.

Two 130 g batches of SZ were prepared as described above. The preparation of both batches included freeze drying of the Zirconium Oxide cake with colloidal silica added, and sulphation by adding about 0.5 M H₂SO₄ to the filtration cake and heating at about 110° C. for about 16 hours. One batch, used to prepared SZ-silica, was calcinated as described above. For the second batch, used to prepare FeMnSZ-silica, the dried and sulphated cake was impregnated with a solution comprising a mixture of sufficient Fe(NO₃)₃ and Mn(NO₃)₃ to provide 1.5 wt % in Fe and 0.5 wt % in Mn, respectively. This was followed by heating at about 110° C. for about 16 hours and calcination, as described above.

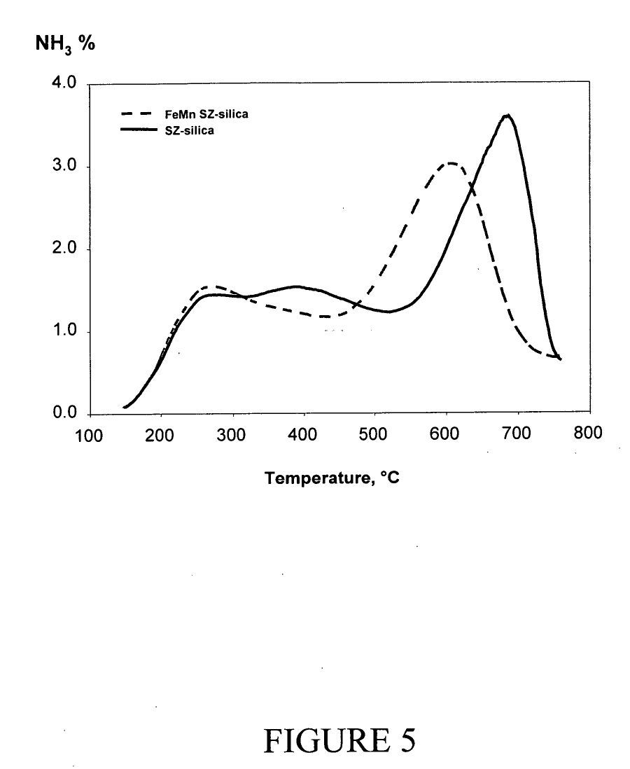

Acidity was assessed by measuring the temperature program desorption (TDP) of NH₃, using conventional instrumentation (Atochem 2910, Micromeritics Instrument Corp., Norcross, Ga.) and methods (1.0 graom sample, 20 ml.min helium, 150 to 750° C. at 10° C./min remperature ramp). The TDP curves for SZ-silica and FeMnSZ-silica are shown in FIG. 5. For SZ-silica, there was a medium acidity peak at about 395° C., and a strong acidity peak at about 690° C. For FeMnSZ-silica, there was a strong acid peak at about 610° C.

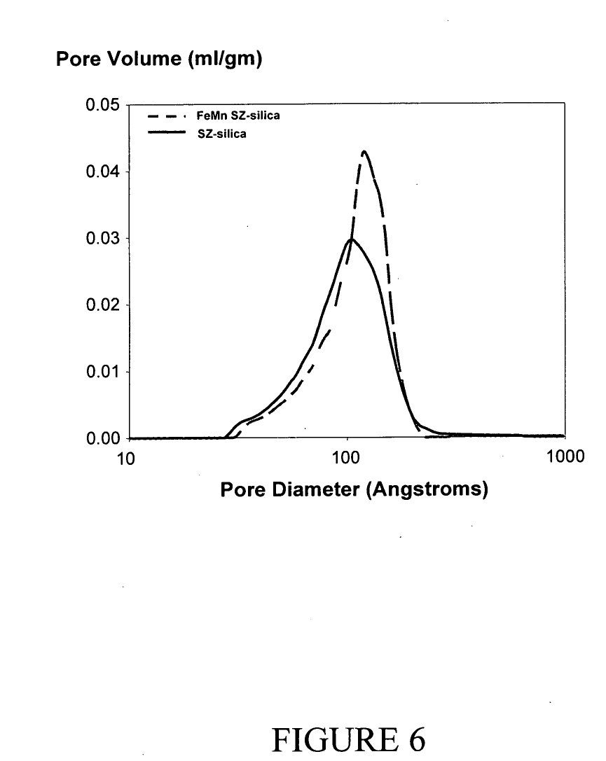

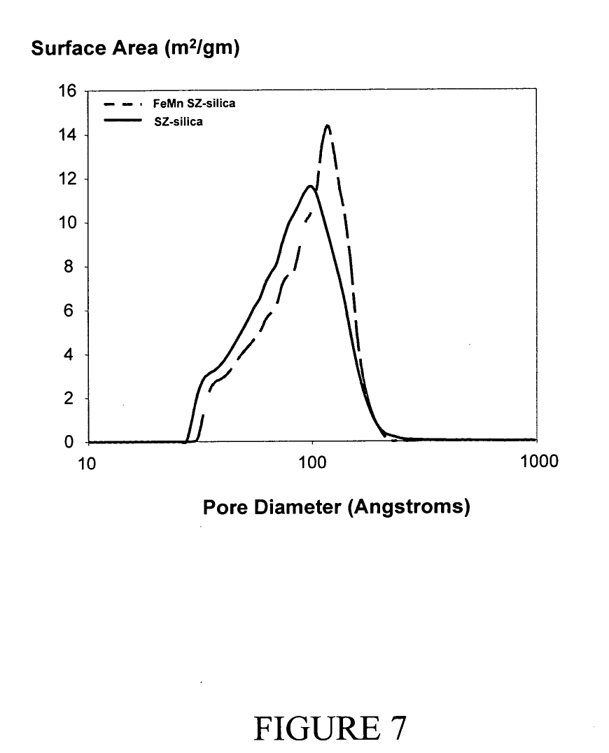

In addition, the pore size distribution of SZ-silica and FeMnSZ-silica were compared using the BET and DFT PLUS, as described above. SZ-silica had similar surface area and pore volume characteristics as previous preparations. FeMnSZ-silica had a surface area of 143.2 m²/gm and a pore volume of about 0.34 ml/g. And, as shown in FIGS. 6 and 7, FeMnSZ-silica had a broader pore volume and surface area distribution than SZ-silica. FeMnSZ-silica also had a peak pore volume at a PD of about 105 Angstroms, while SZ-silica a peak pore volume at a PD of about 118 Angstroms. Likewise, FeMnSZ-silica had a peak surface area at a PD of about 99 Angstroms, while SZ-silica had a peak pore volume at a PD of about 118 Angstroms.

Although the present invention has been described in detail, those skilled in the art should understand that they can make various changes, substitutions and alterations herein without departing from the spirit and scope of the invention.

1-10. (canceled)

1. A catalyst prepared by the process comprising:
freeze drying an intermediary of a catalyst.

12. The catalyst as recited in claim 11, wherein said catalyst comprises a Group IV oxide having a surface area of greater than about 40 m²/g and a pore volume of at least about 0.10 ml/g.

13. The catalyst as recited in claim 11, wherein said catalyst comprises an anion-modified Group IV oxide having a surface area of greater than about 60 m²/g and a pore volume of at least about 0.11 ml/g.

14. The catalyst as recited in claim 11, further including maintaining said intermediary at about 110° C. for about 16 to about 24 hours following said freeze drying.

15. The catalyst as recited in claim 14, wherein said catalyst comprises a Group IV oxide having a surface area of greater than about 73 m²/g and a pore volume of at least about 0.23 ml/g.

16. The catalyst as recited in claim 14, wherein said catalyst comprises an anion-modified Group IV oxide containing a refractory mineral and having a pore volume of at least about 0.27 ml/g.

17. A catalyst having a peak ammonia desorption of greater than about 500° C.

18. The catalyst as recited in claim 17 wherein said peak ammonia desorption is greater than about 600° C.

19. The catalyst as recited in claim 17 comprises an anion-modified Group IV oxide.

20. The catalyst as recited in claim 19 wherein said catalyst further includes a metal promoter.

21. The catalyst as recited in claim 17 wherein said catalyst further having a surface area of greater than about 140 m²/g and a pore volume of at least about 0.30 ml/g.

22-23. (canceled)

24. A petrochemical process comprising:
providing an intermediate of a catalyst composition adapted for use in a petrochemical process;
freeze drying the intermediate to form the catalyst composition; and
contacting the catalyst composition with a petrochemical feedstock.

25. The process of claim 24, wherein the petrochemical process is selected from isomerization, alkylation, oligomerization, dehydration, hydrocracking and combinations thereof.

26. The process of claim 24, wherein the petrochemical process comprises isomerization.

27. The process of claim 26, wherein the petrochemical feedstock is selected from C₉ or less paraffins, C₉ or less cyclic hydrocarbons and combinations thereof.

28. The process of claim 24, wherein the petrochemical feedstock comprises a hydrocarbon.

29. The process of claim 24 further comprising calcining the intermediary to form a catalyst composition comprising a surface area of at least about 40 m2/g and a pore volume of at least about 0.10 ml/mg.

30. The process of claim 24 further comprising calcining the intermediary to form a catalyst composition comprising a peak ammonia desorption of at least 500 C.

31. The process of claim 24 further comprising freezing the intermediate prior to freeze drying.

32. The process of claim 24, wherein the catalyst composition comprises an anion-modified Group IV oxide comprising a refractory mineral.

33. The process of claim 24, wherein the intermediary comprises a Group IV salt deposited into a support selected from silica, alumina, clay, magnesia, zeolite, active carbon, gallium, titanium, thorium, boria and combinations thereof.

34. The process of claim 24 further comprising contacting a basic solution with a Group IV salt containing solution to precipitate a Group IV salt to form the intermediate, wherein the Group IV salt comprises a pH of at least 6.
